(19) United States
(12) Patent Application Publication
(10) Pub. No.: US 2008/0119822 A1
(43) Pub. Date: May 22, 2008

(54) ENTERAL FLUID DELIVERY SYSTEM AND METHOD FOR OPEATING THE SAME

(75) Inventor: Christopher A. Knauper, O'Fallon, MO (US)

Correspondence Address: TYCO HEALTHCARE - EDWARD S. JARMOLOWICZ, 15 HAMPSHIRE STREET, MANSFIELD, MA, 02048, US

(73) Assignee: TYCO HEALTHCARE GROUP LP, MANSFIELD, MA (US)

(21) Appl. No.: 11/561,308

(22) Filed: Nov. 17, 2006

(51) Int. Cl. A61M 31/00

(52) U.S. Cl. 604/516; 604/151

(57) ABSTRACT

An enteral fluid delivery system includes a pump unit that includes a motor coupled to a rotor. The rotor is configured to receive a portion of a pump tube. The motor drives the rotor to pump enteral feed and flush fluids through the pump tube during feed and flush cycles, respectively. A processing unit is operatively connected to the pump unit and controls the pump unit during the feed and flush cycles. The processing unit is programmable to vary a flushing fluid flow rate at which the rotor pumps the flush fluid during a flush cycle. A user interface is operatively connected to the processing unit and enables a user to select between at least two different non-zero flushing fluid flow rates.

### BACKGROUND OF THE INVENTION

This invention relates to enteral fluid delivery, and more particularly, to enteral fluid delivery systems and methods for operating enteral fluid delivery systems.

An enteral fluid delivery system generally includes a fluid delivery tube, sometimes referred to as a feeding tube, that is connected to a patient for delivering nutrients and/or medication to the patient enterally. Feeding tubes are typically flushed with flushing liquid, most commonly water, to ensure that a required amount of medicine is delivered and to avoid obstruction of the feeding tube. Flushing is also used to unclog any blockage in the tube. Obstruction of the feeding tube is usually caused by feeding formula or certain types of medication left inside of the tube. Once a feeding tube becomes blocked, an undesirable amount of time, effort and resources are required to unclog the feeding tube. Sometimes, when the blockage is excessive, replacement of the tube is the only way to continue the treatment which causes patients to undergo the unnecessary pain of insertion of a new feeding tube. Therefore, blockage of feeding tubes can pose a health hazard to patients and add additional healthcare cost.

Conventional enteral fluid delivery systems are provided with at least one feeding volume and at least one feeding flow rate. The feeding volume and feeding flow rate can be selected, or programmed, by an attending physician to allow for different feeding schedules. The physician is afforded the opportunity to program different feeding volumes and feeding flow rates based upon the individual patient, the amount and type of nutrient and/or medication to be delivered, and the like. Following one or more feeding operations, the feeding tube is flushed. The feeding tube is flushed with fluid at a flush flow rate set by the manufacturer. The flush flow rate is not programmable, but instead is preset at the time of manufacture. Certain fluid delivery systems have the flush flow rate set at manufacture to equal a feeding flow rate.

However, conventional fluid delivery systems have experienced limitations. In certain applications, the manufacturer set flush flow rate is not desirable. For example, when the flush flow rate is preset to equal a slow feeding flow rate, the flush flow rate may be too slow to adequately clear blockage of the feeding tube. Also, slow flush rates may require excessive time to complete a flush operation. Alternatively, when the flush flow rate equals a fast feeding flow rate, it may cause patient discomfort or pose a health hazard. For example, a patient's stomach may become distended because too much fluid has been delivered too quickly, which may cause discomfort. A patient may also develop edema due to excess liquid intake. In addition, an unduly high flushing flow rate may result in rupture of the feeding tube. Conventional fluid delivery systems do not afford the ability to plan a flushing operation, and thus lack the ability to account for the age and size of the patient, the patient's condition, the need and restriction for the liquid intake and the size of the feeding tube.

A need remains for an improved enteral fluid delivery system that addresses the above concerns and overcomes other disadvantages experienced heretofore.

### BRIEF DESCRIPTION OF THE INVENTION

In accordance with one aspect, an enteral fluid delivery system is provided that includes a pump unit having a motor coupled to a rotor. The rotor is configured to receive a portion of a pump tube. The motor drives the rotor to pump enteral feed and flush fluids through the pump tube during feed and flush cycles, respectively. A processing unit is operatively connected to the pump unit and controls the pump unit during the feed and flush cycles. The processing unit is programmable to vary a flushing fluid flow rate at which the rotor pumps the flush fluid during a flush cycle. A user interface is operatively connected to the processing unit and enables a user to select between at least two different non-zero flushing fluid flow rates.

In accordance with another aspect, a computer readable medium is provided for use by an enteral fluid delivery system including a user interface, a processing unit, and a pump unit operative to pump enteral feed and flush fluids through a pump tube during feed and flush cycles, respectively. The processing unit is operatively connected to the pump unit. The medium includes instructions directing the processing unit to control operation of the pump unit during the feed and flush cycles. The medium also includes instructions directing the processing unit to vary a flushing fluid flow rate at which the pump unit pumps the flush fluid during a flush cycle, and instructions directing the processing unit to control the user interface to enable a user to select between at least two different non-zero flushing fluid flow rates.

In accordance with another aspect, a method is provided for operating an enteral fluid delivery system including a user interface and a pump unit operative to pump flush and enteral feed fluids through a pump tube. The method includes pumping an enteral feed fluid through the pump tube to deliver the enteral feed fluid to a patient during a feed cycle. Options are provided, at the user interface, to select between at least two different non-zero flushing fluid flow rates, at which the pump unit pumps the flush fluid through the pump tube during a flush cycle. The method also includes pumping the flush fluid through the pump tube at the selected flushing fluid flow rate during the flush cycle.

### BRIEF DESCRIPTION OF THE DRAWINGS

FIG. 1 is a block diagram of an exemplary enteral fluid deliver system formed in accordance with an embodiment of the present invention;

FIG. 2 is a block diagram of certain functional units within the system of FIG. 1.

FIG. 3 illustrates exemplary alternative amplitude modulated and pulse width modulated signals that may be supplied during a flush cycle.

FIG. 4 is an exemplary screen shot for an opening menu that may be presented in connection with programming a continuous feed and flush mode.

FIG. 5 is an exemplary screen shot for an alternative opening menu that may be presented in connection with an intermittent feed and flush mode.

FIG. 6 is an exemplary screen shot of an adjust flush menu that may be presented when the user selects an adjust flush option from either of the menus shown in FIGS. 4 and 5.

FIG. 7 is an exemplary screen shot of a flush rate menu that may be presented when the user selects a flush rate option in the adjust flush rate menu shown in FIG. 6.

FIG. 8 is an exemplary screen shot of a flush volume menu that may be presented when the user selects a flush volume option in the adjust flush rate menu shown in FIG. 6.

FIG. 9 is an exemplary screen shot of a flush interval menu that may be presented when the user selects a flush interval option in the adjust flush rate menu shown in FIG. 6.

FIG. 10 is a flowchart illustrating an exemplary processing sequence for operating the enteral fluid delivery system shown in FIGS. 1 and 2.

### DETAILED DESCRIPTION OF THE INVENTION

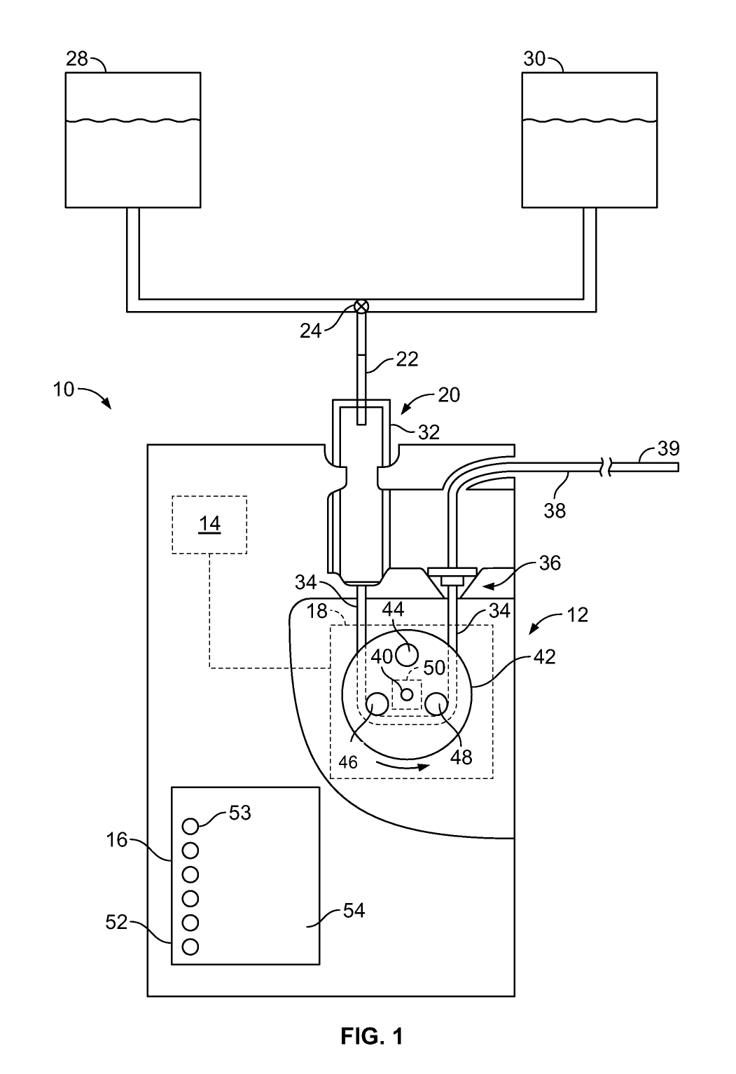

FIG. 1 is a block diagram of an exemplary enteral fluid delivery system 10 formed in accordance with an embodiment of the present invention. The system 10 generally includes a pump unit 12 for pumping enteral fluids, such as but not limited to, enteral medication, feed, and flush fluids, during medication, feed, and flush cycles, respectively. The system 10 also includes a processing unit 14 operatively connected to the pump unit 12 for controlling the pump unit 12 during the feed and flush cycles. A user interface 16 is operatively connected to the processing unit 14 to enable a user to control operation of the system 10.

The pump unit 12 includes a motor 18 and a fluid delivery set 20. The fluid delivery set 20 includes an inlet tube 22 having an inlet 24 coupled in fluid communication with feed and flush fluid sources 28 and 30, respectively. The feed and flush fluid sources 28 and 30, respectively, may each be any suitable type of source, such as, but not limited to, a flexible bag or rigid container. Alternatively, the inlet tube 22 may only be coupled in fluid communication with a flush fluid source 30 or may be coupled in fluid communication with a source of medicine. The inlet tube 22 is joined to a drip chamber 32 that is secured to one end of a pump tube 34. The pump tube 34 includes a flexible portion which may be fabricated from any suitable material(s) enabling the pump tube 34 to function as described herein, such as, but not limited to, plastic. A fluid delivery tube 38 is coupled in fluid communication with an outlet 36 of the pump tube 34 and supplies fluid pumped by the system 10 to an enteral feeding tube 39 connected to a patient (not shown) or to another medical fluid delivery system (not shown).

The pump unit 12 may include any suitable component(s), configuration(s), and/or arrangement(s) that enable the pump unit 12 to function as described herein. In the exemplary embodiment, the motor 18 includes a rotor shaft 40 coupled to a rotor 42. The rotor 42 receives the flexible portion of the pump tube 34 such that the pump tube 34 interacts with rollers 44, 46, 48 mounted on the rotor 42 to form a peristaltic pump. Rotation of the rotor 42 in the direction indicated by the arrow in FIG. 1 causes the rollers 44, 46, 48 to interact with pump tube 34 and pump fluid through the pump tube 34 at a rate which is determined by the rotation rate of rotor 42. Although three rollers 44, 46, 48 are illustrated, the rotor 42 may include any number of rollers enabling the rotor 42 to function as described herein. The rotor shaft 40 may optionally be coupled to the rotor 42 using a gear assembly 50 which includes one or more gears for varying a speed of the rotor shaft 40 relative to the rotor 42. The motor 18 may be any suitable type of motor enabling the component(s) of system 10, and/or system 10 as a whole, to function as described herein. For example, the motor 18 may optionally be a D.C. motor.

The processing unit 14 controls operation of the pump unit 12 to control feed and flush fluids pumped through the pump tube 34 to the fluid delivery tube 38 during feed and flush cycles, respectively. Each cycle includes pumping fluid through the pump tube 34 until a predetermined volume of fluid has been delivered through the feed delivery tube 38. The processing unit 14 may be programmable, at the user interface 16, to control the volume of fluid, the rate of fluid, and/or length of time that fluid is delivered through the fluid delivery tube 38. The user interface 16 may also be used to program a schedule of feeding and flushing cycles. Specifically, the processing unit 14 is programmable to vary a flushing fluid flow rate at which the pump unit 12 pumps flush fluid through the pump tube 34 during a flush cycle. The processing unit 14 may be programmable, at the user interface 16, to pump flush fluid at any suitable number of different non-zero flushing fluid flow rates, where the flow rate is programmed to any suitable value, depending upon the desirable operable range of the flushing fluid flow rate. Moreover, the processing unit 14 may be programmable, at the user interface 16, to pump any suitable number of different non-zero flushing fluid volumes for a flush cycle, wherein each volume has any suitable value, depending upon the desirable operable range of the flushing fluid volume.

The processing unit 14 varies the flushing fluid flow rate at which the rotor 42 rotates by varying a speed of the motor 18. For example, the processing unit 14 may operate the motor 18 at slow and/or fast speeds during different flush cycles to carry out different flushing fluid flow rates. Optionally, the processing unit 14 may operate the motor 18 at two or more different speeds during portions of a single flush cycle. The processing unit 14 may control the speed of the motor 18 by adjusting a voltage applied to the motor 18 (e.g., 6V, 12V, 14V, 24V, and the like). For example, the motor 18 may be driven at 6 volts to achieve a slow flushing fluid flow rate, or at 14 or 24 volts to achieve a fast flushing fluid flow rate. Alternatively, the processing unit 14 may control the speed of the motor 18 through pulse width modulation. For example, the motor 18 may be driven with a series of drive pulses, where a frequency and/or width of each drive pulse is modulated to adjust the rotation speed of the motor 18. Optionally, the processing unit 14 may be programmable to operate the motor 18 at any suitable number of different speeds between fast and slow speeds.

In addition or alternatively, the processing unit 14 may vary the flushing fluid flow rate by operating the motor 18 intermittently during portions of a flush cycle, rather than continuously. During intermittent operation, the motor 18 is deactivated such that the rotor shaft 40 becomes stationary during one or more intervals in the flush cycle. The number of intermittent active intervals and/or a length of time between each intermittent active interval of the motor 18 may be set at the user interface 16. For example, the length of time between active intervals of the motor 18 may be equal during a single flush cycle. Alternatively, the active intervals of the motor 18 during a single flush cycle may differ in length.

The processing unit 14 may be programmed in any language(s), manner(s), fashion(s), arrangement(s) and/or configuration(s), and/or may include any component(s) (e.g., a memory), that enable the processing unit 14 to function as described herein. For example, the processing unit 14 may be programmed using instructions recorded on a computer readable medium.

The user interface 16 is operatively connected to the processing unit 14 to enable a user to control operation of the system 10. The user interface 16 may be any suitable type of interface having any suitable component(s) that enable the user interface 16 to function as described herein. Examples of some suitable user interface components include, but are not limited to, displays, keyboards, trackball, buttons, mice, and/or touch screens. In the exemplary embodiment, the user interface 16 includes a display 52 having a touch screen 54 that enables a user to select from a plurality of options displayed on the display 52. One or more rows of buttons 53 may be provided along the side, top, and/or bottom of the touch screen 54. The buttons 53 may have different functions depending upon the mode and menu presented.

The user interface 16 may include options that enable a user to turn the system 10 on and off, initiate a feed cycle, initiate a flush cycle, as well as schedule one or more feed and/or flush cycles. The user interface 16 may also include options enabling a user to select a feeding fluid flow rate for a feed cycle, a total feeding fluid volume for a feed cycle, a flushing fluid flow rate for a flush cycle, a total flushing fluid flow volume for a flush cycle, a length of time for a feed cycle, and/or a length of time for a flush cycle. The user interface 16 may also include options enabling a user to schedule a plurality of feed and/or flush schedules over time, for example including an interval between feed and/or flush cycles. The user interface 16 may enable a user to select between any number of different non-zero flushing fluid flow rates, each having any suitable value, depending upon the desirable operable range of the flushing fluid flow rate. Moreover, the user interface 16 may enable a user to select between any number of different non-zero flushing fluid flow volumes for flush cycle, each having any suitable value, depending upon the desirable operable range of the flushing fluid flow volume. The user interface 16 may enable a user to select between actual flushing fluid flow rates, actual motor speeds, and/or a number of, and/or length of time between each, intermittent operation of the motor 18. When scheduling a plurality of feed and/or flush cycles over time, the user interface 16 may include options that enable a user to select various parameters, such as, but not limited to those described in this paragraph, for each of the feed and flush cycles being scheduled. Using the user interface 16, a plurality of feed and flush cycles may be programmed by the user to be performed automatically by the processing unit 14 or some or all of the cycles may be selected as being initiated by a user. The user interface 16 may optionally give a visual and/or audible indication of when a feed and/or flush cycle is automatically beginning or ending, and/or may optionally give a visual and/or audible indication to a user that a cycle is due to be initiated.

To facilitate operation of the system 10, as well as monitoring of the feed and flush cycles, the display 52 of the user interface 16 may display various screens and parameters of the system 10, such as, but not limited to, an indication of a current flush or feed cycle being performed, a selected feeding and/or flushing fluid flow rate, a selected feeding and/or flushing fluid flow volume, a feed schedule over time, a flush schedule over time, and/or an interval between feed and/or flush cycles.

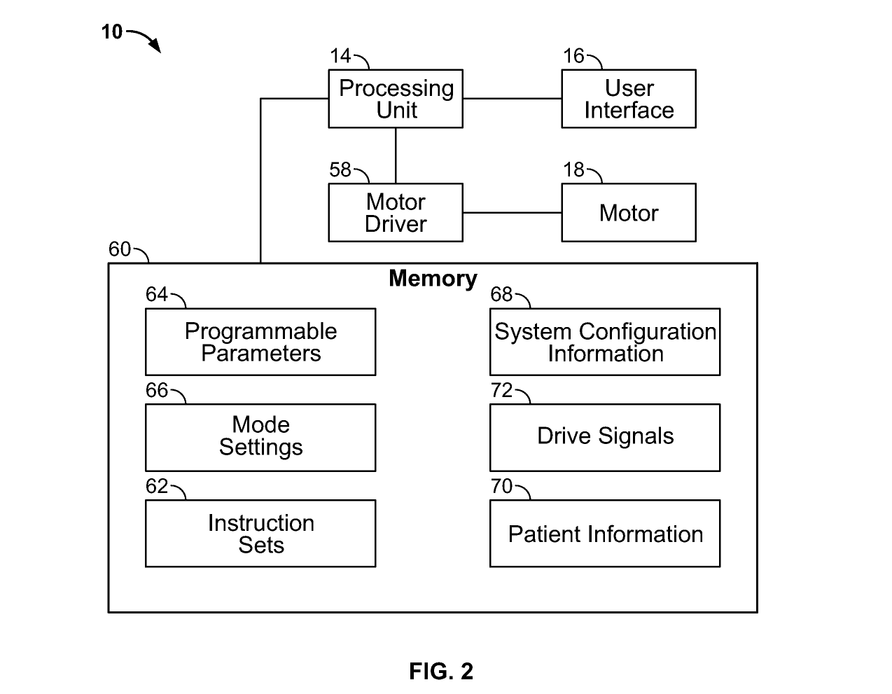

FIG. 2 is a block diagram of certain functional units within the system 10 of FIG. 1, namely the user interface 16, processing unit 14, the motor 18, a motor driver 58 and memory 60. The memory 60 represents a computer readable medium that stores instruction sets 62 to direct the processing unit 14 regarding operation of the system 10. The memory 60 also stores programmable parameters 64, such as, but not limited to, feed rate and volume, flush rate and volume, and feed and flush intervals mode. The memory 60 also stores settings 66 such as, but not limited to, intermittent and/or continuous modes. The memory 60 also stores system configuration information 68, such as, but not limited to, the voltage and current levels that are available to drive motor 18 and the gear box configurations (if any) that are available to achieve different rotor speeds. The memory 60 may also store patient information 70, such as a patient's name, age, weight, and/or health condition, along with the feed and flush parameters implemented for the patient. The patient information 70 may also retain a history of the feed and flush cycles administered to the patient. During the initial configuration of the system 10, the system configuration information 68 is loaded. For example, voltage and current levels may be loaded, identifying the capabilities of the motor 18. The motor driver 58 is controlled by the processing unit 14 to supply drive pulses to the motor 18 at a predetermined voltage or current. The drive pulses may be stored as digital pulse width modulated and/or amplitude modulated sequences.

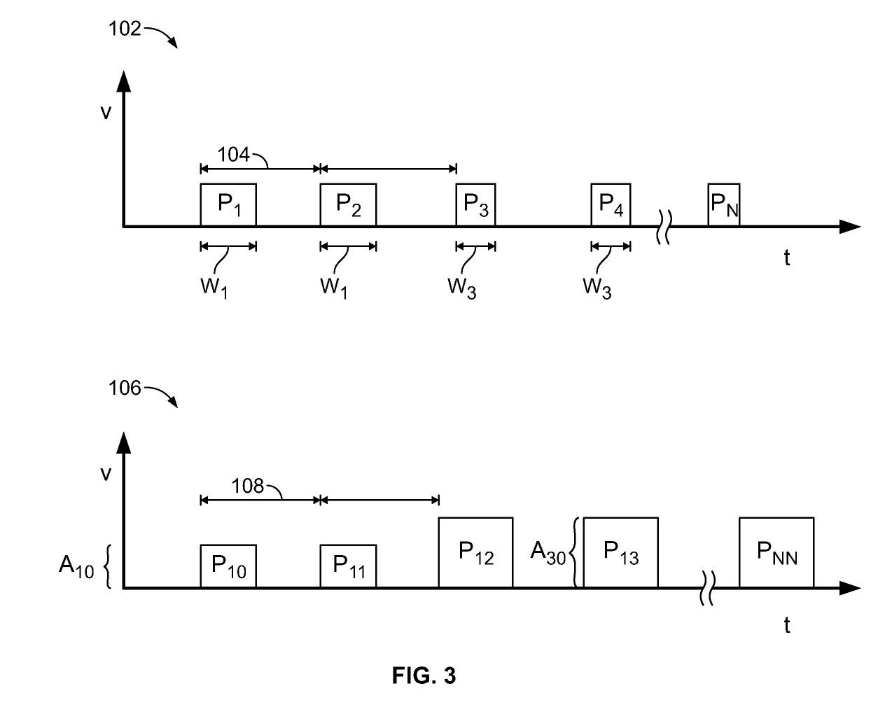

FIG. 3 illustrates exemplary amplitude and pulse width modulated (PWM) signals that may be supplied by the motor driver 58 (shown in FIG. 2) during a flush cycle. Drive signal 102 represents a portion of a PWM signal that is supplied during a flush cycle. The drive signal 102 includes pulses P₁ to Pₙ that have a common amplitude and are separated by a period 104. The initial series of pulses P₁ and P₂ have a width W₁ that is twice the width W₃ of the next series of pulses P₃ and P₄. The drive motor 58 may adjust the pulse width from W₁ to W₃ to drive the motor 18 faster during an initial portion of a flush cycle (e.g., to fill the tube with flush fluid) and slower during a remainder of the flush cycle.

Drive signal 106 represents a portion of an amplitude modulated signal that is supplied during a flush cycle. The drive signal 106 includes pulses P₁₀ to Pₙₙ that have a common width and are separated by a period 108. The initial series of pulses P₁₀ and P₁₁ have an amplitude A₁₀ that is half the amplitude A₃₀ of the next series of pulses P₁₂ and P₁₃. The amplitude A₁₀ may initially be set low when it is desirable to begin a flush cycle at a slower rate, followed by a faster flush rate and higher amplitude A₃₀.

Once the system 10 is configured and the patient's programmable parameters 64 are set, but before operation, the processing unit 16 may create one or more sequences of digital drive signals 72 to be applied subsequently during feed cycles and one or more drive signals to be applied subsequently during flush cycles. Once created, the drive signals 72 (FIG. 2) are stored in memory 60. Alternatively, the drive signals 72 need not be stored, but instead the processing unit 16 may generate the drive signals 72 in real time during feed and flush operations.

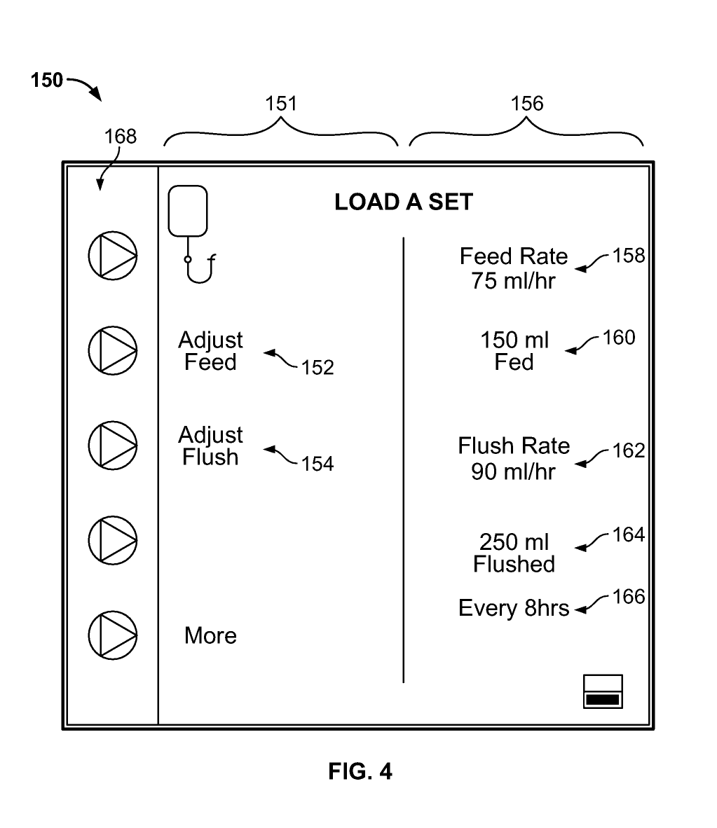

Next, a series of exemplary screen shots will be described to program various parameters in connection with FIGS. 4-9.

FIG. 4 is an exemplary screen shot for an opening menu 150 that may be presented in connection with programming a continuous feed and flush mode. The opening menu 150 includes a button control portion 151 having an adjust feed option 152 and an adjust flush option 154. The opening menu 150 also has a display portion 156 that illustrates feed and flush parameters that are set to exemplary values. The parameters include feed rate 158, feed volume 160, flush rate 162, flush volume 164 and flush interval 166. The opening menu 150 includes a series of buttons 168 that allow the user to select various options and navigate between menu screens.

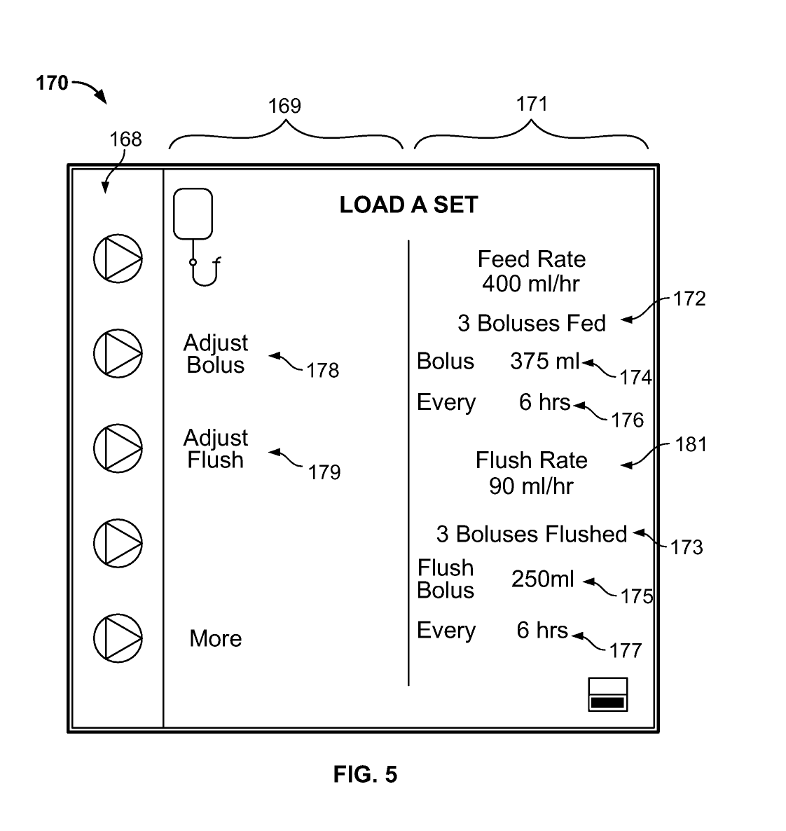

FIG. 5 is an exemplary screen shot for an alternative opening menu 170 that may be presented in connection with an intermittent feed and flush mode. The menu 170 has a button control portion 169 and a display portion 171. The opening menu 170 differs from the opening menu 150, among other things, by displaying a feed bolus count 172, a volume 174 per feed bolus, and a frequency 176 at which each feed bolus is to be delivered. The opening menu 170 differs from the opening menu 150, among other things, by displaying a flush bolus count 173, a volume 175 per flush bolus, and a frequency 177 at which each flush bolus is to be delivered. The opening menu 170 also displays an adjust bolus option 178, as well as an adjust flush option 179 and the flush rate 181. Alternatively, the opening menu 170 may display an intermittent feed mode with a continuous flush mode or an intermittent flush mode with a continuous feed mode.

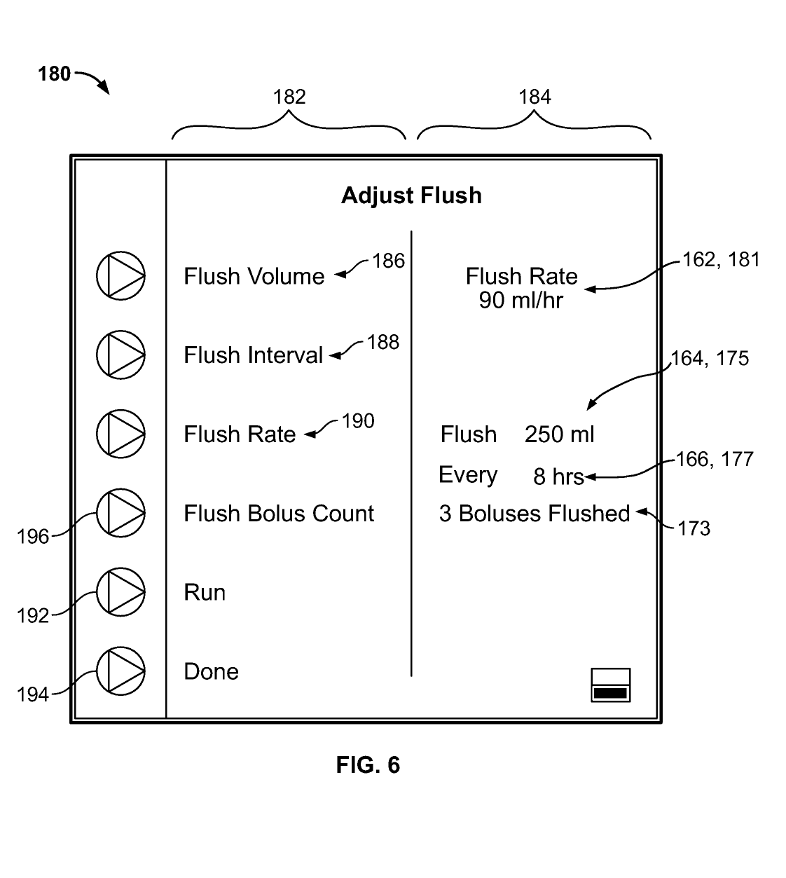

FIG. 6 is an exemplary screen shot of an adjust flush menu 180 that may be presented when the user selects the adjust flush option 154, 179 in either of the menus 150 and 170 (shown in FIGS. 4 and 5, respectively). The adjust flush menu 180 is divided into a flush control portion 182 and a flush parameter portion 184. The flush control portion 182 includes a flush volume option 186, a flush interval option 188, a flush rate option 190, a run option 192, and a done option 194. The flush control portion 182 may also include a flush bolus count option 196 for programming the number of boluses flushed during an intermittent flush mode. The flush parameter portion 184 illustrates the values for the flush rate 162, 181, the flush volume 164, 175, and the flush frequency 166, 177 that are presently programmed. The flush parameter portion 184 may also illustrate a value for the flush bolus count 173. When the user is satisfied with the flush parameters, the done option 194 may be selected and control returns to the prior menu screen (e.g., the opening menus 150 or 170, an adjust feed menu, and the like). When the user desires to initiate operation of the system 10, the user selects the run option 192.

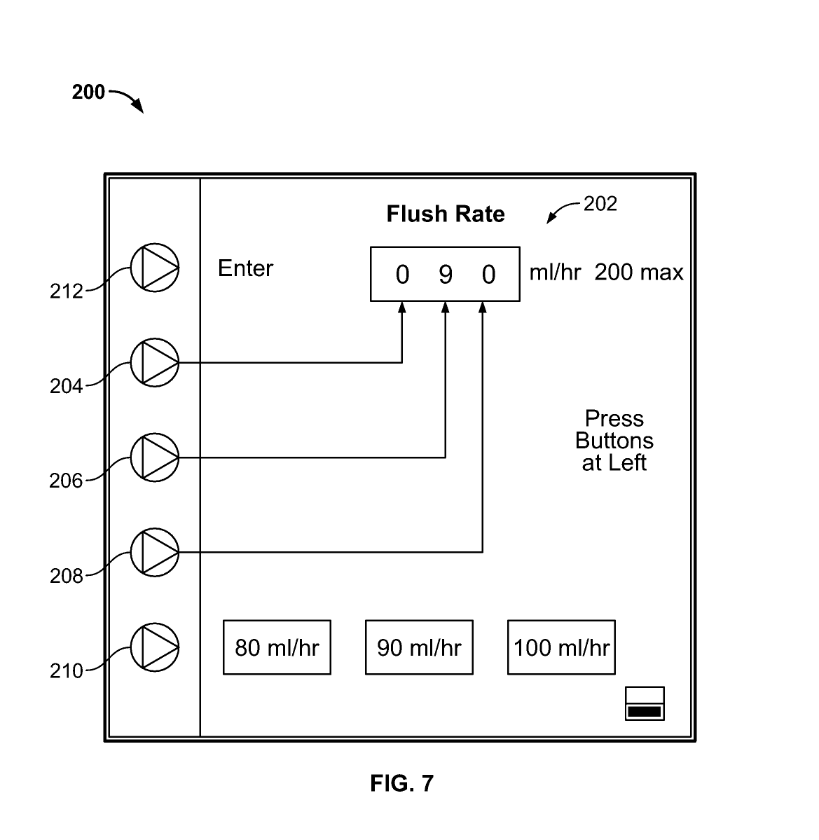

When the user desires to change one of the flush rate, volume, or frequency, the corresponding one of the flush rate, volume and frequency 190, 186, and 188, is selected. When the flush rate 190 is selected, a flush rate menu 200 in FIG. 7 is presented. The flush rate menu 200 may include a rate adjustment window 202. Buttons 204, 206, and 208 are pressed successively to increment through values (e.g., ones, tens, hundreds) for the flush rate. The digits cycle from 0 to 9 and then return to 0 as the corresponding button 204-208 is pressed. Additionally or alternatively, the flush rate menu 200 may include a button 210 for cycling through two or more preset flush rate values. Any suitable indication, such as, but not limited to, a symbol or highlighting, may be displayed to indicate which preset flush rate has been selected. Once the desired flush rate is displayed or selected from the preset values, the enter button 212 is selected and the displayed flush rate is entered as the programmed flush rate.

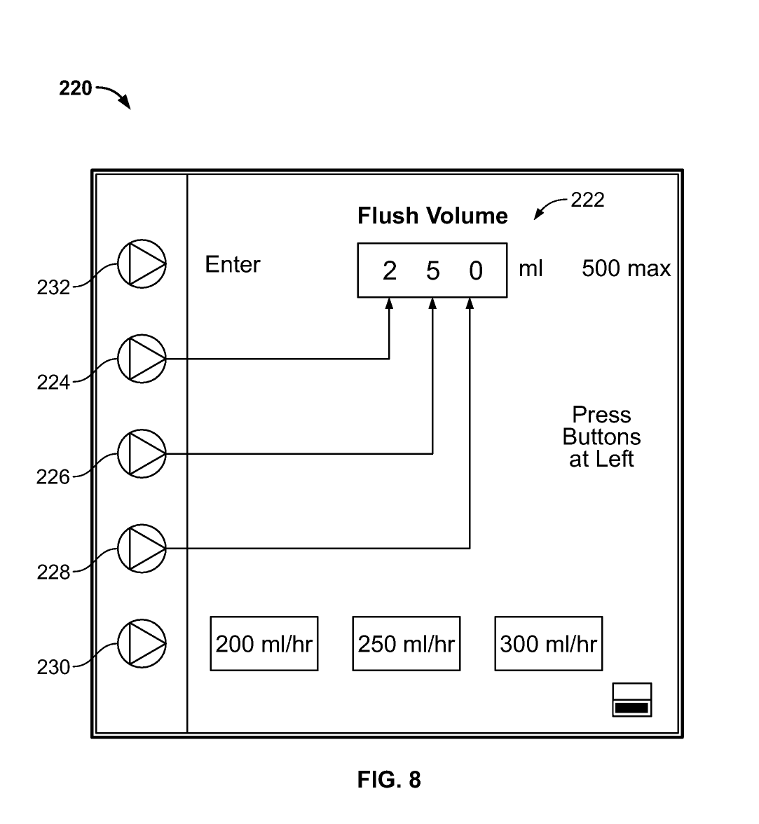

When the flush volume 186 (shown in FIG. 6) is selected, a flush volume menu 220 in FIG. 8 is presented. The flush volume menu 220 may include a volume adjustment window 222. Buttons 224, 226, and 228 are pressed successively to increment through values (e.g., ones, tens, hundreds) for the flush volume. The digits cycle from 0 to 9 and then return to 0 as the corresponding button 224-228 is pressed. Additionally or alternatively, the flush volume menu 220 may include a button 230 for cycling through two or more preset flush volume values. Any suitable indication, such as, but not limited to, a symbol or highlighting, may be displayed to indicate which preset flush volume has been selected. Once the desired flush volume is displayed or selected from the preset values, the enter button 232 is selected and the displayed flush volume is entered as the programmed flush volume.

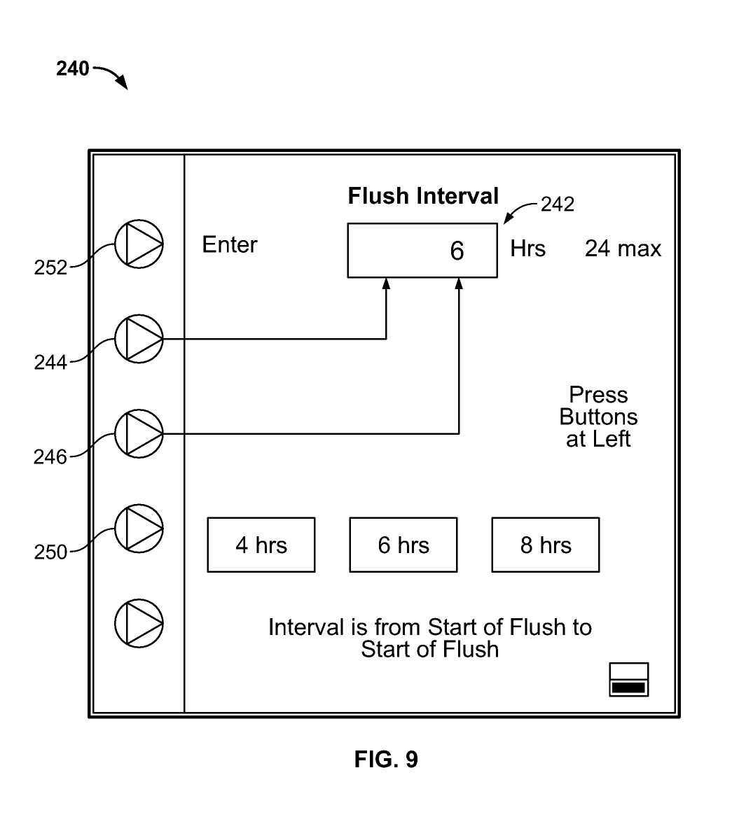

When the flush interval 188 (shown in FIG. 6) is selected, a flush interval menu 240 in FIG. 9 is presented. The flush interval menu 240 may include an interval adjustment window 242. Buttons 244 and 246 are pressed successively to increment through values (e.g., ones, tens) for the flush interval. The digits cycle from 0 to 9 and then return to 0 as the corresponding button 244-246 is pressed. Additionally or alternatively, the flush interval menu 240 may include a button 250 for cycling through two or more preset flush interval values. Any suitable indication, such as, but not limited to, a symbol or highlighting, may be displayed to indicate which preset flush interval has been selected. Once the desired flush interval is displayed or selected from the preset values, the enter button 252 is selected and the displayed flush interval is entered as the programmed flush interval.

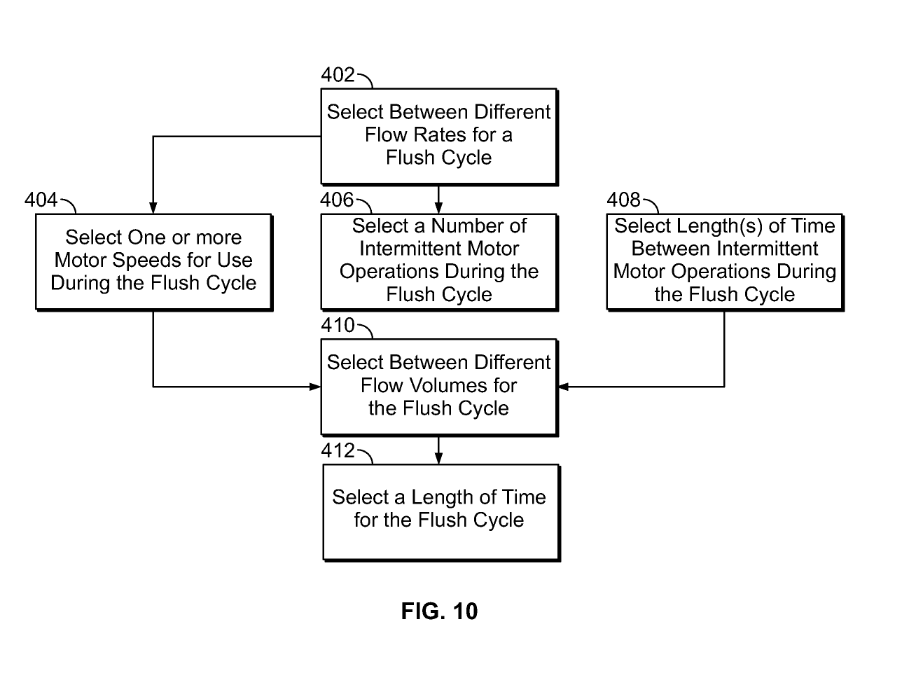

FIG. 10 is a flowchart illustrating an exemplary processing sequence for operating an enteral fluid delivery system, such as, but not limited to, the system 10 (shown in FIGS. 1 and 2). The method 400 includes selecting 402 between two or more different non-zero flushing fluid flow rates for a flush cycle, for example using the user interface 16 (shown in FIGS. 1 and 2). Selecting 402 between two or more different non-zero flushing fluid flow rates may include selecting 404 one or more motor speeds for use during one or more portions of the flush cycle, selecting 406 a number of intermittent operations of the motor 18 (shown in FIGS. 1 and 2) during the flush cycle, and/or selecting 408 one or more lengths of time between intermittent operations of the motor 18 during the flush cycle. The method 400 may also include selecting 410 between two or more different non-zero flushing fluid flow volumes for the flush cycle, and/or selecting 412 a length of time for the flush cycle. Once all desired parameters for the flush cycle have been selected, the flush cycle is initiated 412 to pump the flush fluid through the pump tube 34 (FIG. 2) at the selected flushing fluid flow rate and volume.

The enteral fluid delivery system and method described herein enables a user to select from a variety of different non-zero fluid flow rates for a flushing cycle. The system and method may facilitate the selection of a flushing fluid flow rate that is high enough to clear blockage within a fluid delivery tube but is low enough to prevent discomfort and/or injury to a patient connected to the fluid delivery tube.

Exemplary embodiments are described and/or illustrated herein in detail. The embodiments are not limited to the specific embodiments described herein, but rather, components and/or steps of each embodiment may be utilized separately and independently from other components and/or steps described herein. Each component, and/or each step, can also be used in combination with other components and/or steps.

When introducing elements/components/etc. described and/or illustrated herein, the articles “a”, “an”, “the”, “said”, and “at least one” are intended to mean that there are one or more of the element(s)/component(s)/etc. The terms “comprising”, “including” and “having” are intended to be inclusive and mean that there may be additional element(s)/component(s)/etc. other than the listed element(s)/component(s)/etc.

While the invention has been described in terms of various specific embodiments, those skilled in the art will recognize that the invention can be practiced with modification within the spirit and scope of the claims.

What is claimed is:

1. An enteral fluid delivery system, comprising:
a pump unit including a motor coupled to a rotor, the rotor being configured to receive a portion of a pump tube, the motor driving the rotor to pump enteral feed and flush fluids through the pump tube during feed and flush cycles, respectively;
a processing unit, operatively connected to the pump unit, controlling the pump unit during the feed and flush cycles, the processing unit being programmable to vary a flushing fluid flow rate at which the rotor pumps the flush fluid during a flush cycle; and
a user interface, operatively connected to the processing unit, enabling a user to select between at least two different non-zero flushing fluid flow rates.

2. The system of claim 1, wherein the processing unit varies the flushing fluid flow rate by varying a speed of the motor.

3. The system of claim 1, wherein, during the flush cycle, the processing unit varies a speed of the motor between at least two different motor speeds.

4. The system of claim 1, wherein, during a flush cycle, the processing unit intermittently deactivates the motor such that the motor is stationary during a portion of the flush cycle.

5. The system of claim 1, wherein the user interface comprises an option for selecting between the at least two different non-zero flushing fluid flow rates.

6. The system of claim 1, wherein the user interface comprises a touch screen including an option for selecting between the at least two different non-zero flushing fluid flow rates.

7. The system of claim 1, wherein the user interface enables the user to select between at least two non-zero flushing fluid volumes.

8. The system of claim 1, further comprising a fluid delivery set including the pump tube, and an inlet coupled in fluid communication with a source of at least one of the enteral feed and flush fluid.

9. The system of claim 1, further comprising the pump tube, wherein the pump tube comprises a pair of inlets coupled in fluid communication with sources of the enteral feed and flush fluids.

10. The system of claim 1, wherein the motor is coupled to the rotor using a gear assembly.

11. A computer readable medium for use by an enteral fluid delivery system including a user interface, a processing unit, and a pump unit operative to pump enteral feed and flush fluids through a pump tube during feed and flush cycles, respectively, the processing unit operatively connected to the pump unit, the medium comprising:
instructions directing the processing unit to control operation of the pump unit during the feed and flush cycles;
instructions directing the processing unit to vary a flushing fluid flow rate at which the pump unit pumps the flush fluid during a flush cycle; and
instructions directing the processing unit to control the user interface to enable a user to select between at least two different non-zero flushing fluid flow rates.

12. The medium of claim 11, wherein the pump unit includes a motor, the medium further comprising instructions directing the processing unit to vary the flushing fluid flow rate by varying a speed of the motor.

13. The medium of claim 11, wherein the pump unit includes a motor, the medium further comprising instructions directing the processing unit to vary a speed of the motor between at least two different motor speeds during the flush cycle.

14. The medium of claim 11, wherein the pump unit includes a motor, the medium further comprising instructions directing the processing unit to intermittently deactivate the motor during the flush cycle such that the motor is stationary during a portion of the flush cycle.

15. The medium of claim 11, further comprising instructions directing the processing unit to control the user interface to enable the user to select between at least two non-zero flushing fluid volumes.

16. The medium of claim 11, wherein the pump unit includes a motor coupled to a rotor using a gear assembly, the medium further comprising instructions directing the processing unit to adjust the gear assembly to vary the flushing fluid flow rate.

17. A method for operating an enteral fluid delivery system including a user interface and a pump unit operative to pump flush and enteral feed fluids through a pump tube, the method comprising:
pumping an enteral feed fluid through the pump tube to deliver the enteral feed fluid to a patient during a feed cycle;
providing, at the user interface, options to select between at least two different non-zero flushing fluid flow rates, at which the pump unit pumps the flush fluid through the pump tube during a flush cycle; and
pumping the flush fluid through the pump tube at the selected flushing fluid flow rate during the flush cycle.

18. The method of claim 17, wherein pumping the flush fluid through the pump tube at the selected flushing fluid rate comprises operating a motor of the pump unit at at least two different non-zero speeds during the flush cycle.

19. The method of claim 17, wherein pumping the flush fluid through the pump tube at the selected flushing fluid flow rate comprises intermittently deactivating a motor of the pump unit during the flush cycle such that the motor is stationary during a portion of the flush cycle.

20. The method of claim 17, wherein providing, at the user interface, options to select between the at least two different non-zero flushing fluid flow rates further comprises providing a touch screen including the options to select between the at least two different non-zero flushing fluid flow rates.

21. The method of claim 17, further comprising providing, at the user interface, options to select between at least two different non-zero flushing fluid flow volumes.
